SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC

(19) United States
(12) Patent Application Publication
(10) Pub. No.: US 2003/0220257 A1
(43) Pub. Date: Nov. 27, 2003

(54) Treatment of trauma

(76) Inventors: Robbert Benner, Barendrecht (NL); Nisar Ahmed Khan, Rotterdam (NL)

Correspondence Address: TRASK BRITT, P.O. BOX 2550, SALT LAKE CITY, UT, 84110, US

(21) Appl. No.: 10/409,032

(22) Filed: Apr. 8, 2003

Related U.S. Application Data
(63) Continuation-in-part of application No. 10/028,075, filed on Dec. 21, 2001.

(51) Int. Cl. A61K 38/17; A61K 48/00

(52) U.S. Cl. 514/12; 514/44

(57) ABSTRACT

The invention relates to the treatment of trauma and to the treatment of its immunosuppressive effects. The invention provides a method for modulating or treating an immunosuppressive state in a subject comprising providing the subject with a gene-regulatory peptide or functional analogue thereof, in particular wherein the subject has experienced trauma. The invention also provides use of an NF-κB up-regulating peptide or functional analogue thereof for the production of a pharmaceutical composition for the treatment of a counter anti-inflammatory response syndrome.

### CROSS REFERENCE TO RELATED APPLICATION

This application is a continuation-in-part of U.S. patent application Ser. No. 10/028,075, filed Dec. 21, 2001, pending, the content of the entirety of which is incorporated by this reference.

### TECHNICAL FIELD

The current invention relates generally to biotechnology, and, more specifically, to the body's innate way of modulation of important physiological processes and builds on insights reported in PCT International Patent Publications WO99/59617 and WO01/00259 and PCT International Patent Application PCT/NL02/00639, the contents of the entirety of all of which are incorporated by this reference.

### BACKGROUND

In the aforementioned applications, small gene-regulatory peptides are described that are present naturally in pregnant women and are derived from proteolytic breakdown of placental gonadotropins such as human chorionic gonadotropin (hCG) produced during pregnancy. These peptides (in their active state often only at about 4 to 6 amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except to serve as antigen for the immune system), it now emerges that the body in fact routinely utilizes the normal process of proteolytic breakdown of the proteins it produces to generate important gene-regulatory compounds, short peptides that control the expression of the body's own genes. Apparently the body uses a gene-control system ruled by small broken down products of the exact proteins that are encoded by its own genes.

It is long known that during pregnancy the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

It was shown that certain short breakdown products of hCG (i.e., short peptides which can easily be synthesized, if needed modified, and used as pharmaceutical composition) exert a major regulatory activity on pro- or anti-inflammatory cytokine cascades that are governed by a family of crucial transcription factors, the NFκB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFκB-mediated expression of cytokine genes, thereby keeping the immunological responses in the placenta at bay.

### BRIEF SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFκB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFκB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### DETAILED DESCRIPTION OF THE INVENTION

The invention in particular relates to the treatment of trauma and to the treatment of its immunosuppressive effects.

Trauma is an immense, world-wide, socio-economic problem. In the United States, it is the leading cause of death in those under 45 years of age and the resultant morbidity often leads to significant disability. The cost in terms of hospital care, loss of productivity, and emotional stress is staggering. Efforts directed at accident prevention and efficient triage, and to the establishment of properly staffed trauma centers are of paramount importance. Should the badly injured patient survive a serious head injury and/or major hemorrhage, the next threat to life will be infection. Sepsis has been recognized as the most common cause of late death in these patients and accounts for much of the morbidity in those who recover from infection. Specifically, trauma resulting in major damage to (parts of) the body is often seen after traffic accidents, sport accidents, accidents at work, and so on. Another form of trauma, and its related immunosuppression, is often seen after major surgery. The relationship between the nervous and the immune system following trauma is poorly understood and under investigation. Recent reviews have highlighted the complex nature of the tremendous surge of hormone and catecholamine output from the pituitary-adrenal axis following trauma, which may be mediated through the spinal cord along afferent neurons from the site of tissue destruction. Also, often a generalized depression of the immune system exists, and this continues to be an ongoing area of research interest. Virtually all components of the immune response have been found to be depressed following injury including macrophage, lymphocyte, and neutrophil function; delayed type hypersensitivity (DTH) responses, immunoglobulin (Ig) and interferon (IFN) production, and serum opsonic capacity. Serum peptides, which suppress lymphocyte proliferation in vitro, have been defined, and the immunosuppressive role of excessive complement activation has also been recognized. Immune failure occurs early after trauma and the rapidity with which immune function returns to normal may be the best indicator of clinical recovery. Indeed, immediate down-regulation of the immune response may be a protective mechanism for the host, lest too vigorous an early host response creates a catabolic situation incompatible with early survival. Major trauma significantly alters the composition and function of monocytes-macrophages, lymphocytes, and neutrophils. There is a relative monocytosis after severe injury, the monocytes increasing from 10 per cent to over 30 per cent, one week after injury. Also, it was found that a monocytosis peaked on the eleventh day in hospital following trauma and only slowly returned to normal over several weeks. The surface expression of the class II HLA-DR on peripheral blood monocytes was measured in 60 patients and was depressed in most, immediately following severe trauma and during subsequent sepsis. However, when patients were grouped according to clinical outcome (uneventful recovery, major infection, and death) an interesting pattern arose. The percentage of monocytes that expressed the HLA-DR antigen returned to the normal range by one week in the first group, by three weeks in those with major infection, but never in those who eventually died. Thus, HLA-DR expression on monocytes served as a useful marker, or predictor, of clinical outcome in such patients. When monocytes were incubated with bacterial lipopolysaccharide (LPS), those patients who survived had enhanced HLA-DR antigen expression (stimulated towards the normal range), while monocytes from patients who died were relatively resistant to stimulation. As LPS is thought to externalize all preformed antigen to the cell surface, an “exhaustion” of HLA-DR in the patients who died with an accompanying inability to synthesize new HLA-DR is a possibility.

Expression of HLA-DR antigen may correlate with the ability of these cells to present foreign antigen and thus to initiate a specific immune response.

Monocyte HLA-DR expression within 24 hours of hospital admission, assessment of bacterial contamination, age, and injury severity, have been combined to formulate a useful outcome predictive score. Results may be multiplied by simple scaling factors for degree of contamination and monocyte HLA-DR expression. This is the first trauma scoring system to include a valid assessment of host defense processes, and to successfully identify those patients who eventually developed major infection and subsequently died from sepsis. Interestingly, the presence of hypotension and the amount of blood transfused did not correlate with the development of infection and multi-system organ failure; HLA-DR antigen expression was the best discriminator of poor clinical outcome. Failure of host defenses following trauma is indeed multi-factorial and complex. Various studies have demonstrated the following important defects: depressed monocyte HLA-DR expression and serum opsonic capacity; reduced endogenous IFN and IL-2 production; and increased PGE₂ production. However, trials have been slow to be introduced into clinical practice, despite identification of such defects in man an animals, and extensive laboratory work with immunomodulation both in vitro and in the experimental animal. Defining patients at very high risk of infection and multi-system organ failure before either develop, is paramount to the introduction and interpretation of clinical trials in this area.

The invention provides a method of treatment for traumatized patients comprising the specific immune modulation of host defenses, preferably before infection and its attendant complications arise and become established. We believe that immune enhancement, possibly with combination therapy, will become a principle treatment regimen for these patients in the future. Specific modulators are herein provided as well. In one embodiment, the invention provides a method for modulating or treating a subject in an immunosuppressive state, in particular of a traumatized subject believed to be in need thereof comprising providing the subject with a signaling molecule comprising a gene-regulatory peptide or functional analogue thereof wherein the signaling molecule is administered in an amount sufficient to modulate the immunosuppression. The signal molecule is preferably a short peptide, preferably of at most 30 amino acids long, or a functional analogue or derivative thereof. In a much preferred embodiment, the peptide is an oligopeptide of from about 3 to about 15 amino acids long, preferably 4 to 12, more preferably 4 to 9, most preferably 4 to 6 amino acids long, or a functional analogue or derivative thereof. Of course, such a signaling molecule can be longer, for example by extending it (N- and/or C-terminally), with more amino acids or other side groups, which can for example be (enzymatically) cleaved off when the molecule enters the place of final destination. In particular a method is provided wherein the signaling molecule modulates translocation and/or activity of a gene transcription factor. It is particularly useful when the gene transcription factor comprises an NF-κB/Rel protein or an AP-1 protein.

The invention is further explained by the use of the following illustrative examples.

### EXAMPLES

As indicated above, trauma may induce decreased expression of inflammatory cytokines and other immunomodulating mediators, often due to inhibition of NF-κB and AP-1, and in a preferred embodiment the invention provides a method wherein translocation and/or activity of the NF-κB/Rel or AP-1 protein is upregulated. In one embodiment, the peptide is selected from the group of peptides LQG, AQG, LQGV (SEQ ID NO: 1 of the hereby incorporated accompanying SEQUENCE LISTING), AQGV (SEQ ID NO: 2), LQGA (SEQ ID NO: 3), VLPALP (SEQ ID NO: 4), ALPALP (SEQ ID NO: 5), VAPALP (SEQ ID NO: 6), ALPALPQ (SEQ ID NO: 7), VLPAAPQ (SEQ ID NO: 8), VLPALAQ (SEQ ID NO: 9), LAGV (SEQ ID NO: 10), VLAALP (SEQ ID NO: 11), VLPALA (SEQ ID NO: 13), VLPALPQ (SEQ ID NO: 14), VLAALPQ (SEQ ID NO: 15), VLPALPA (SEQ ID NO: 16), GVLPALP (SEQ ID NO: 17), LQGVLPALPQVVC (SEQ ID NO: 18), LPGCPRGVNPVVS (SEQ ID NO: 19), LPGC, MTRV (SEQ ID NO: 20), MTR, VVC. Of clinical and medical interest and value, the present invention provides the opportunity to selectively control NFκB-dependent gene expression in tissues and organs in a living subject, preferably in a primate, allowing upregulating essentially anti-inflammatory responses such as IL-10, and downregulating essentially pro-inflammatory responses such as mediated by TNF-α, nitric oxide (NO), IL-5, IL-1β. The invention thus provides use of a NFκB regulating peptide or derivative thereof for the production of a pharmaceutical composition for the treatment of a traumatized patient, in particular of a human, and provides a method of treatment of a traumatized human being. It is preferred when the treatment comprises administering to the subject a pharmaceutical composition comprising an NFκB up-regulating peptide or functional analogue thereof. The invention for this purpose provides use of a such signaling molecule comprising an NF-κB up-regulating peptide or functional analogue thereof for the production of a pharmaceutical composition for the treatment of a counter anti-inflammatory response syndrome occurring after a traumatic injury of a subject, in particular wherein translocation and/or activity of the NF-κB/Rel protein is upregulated, resulting in stimulating a cascade of cytokine reactions. In one embodiment, the invention is providing a method and means to treat the systemic immunosuppressive reaction to trauma by providing a subject believed to be in need thereof with a pharmaceutical composition comprising an NF-κB down-regulating peptide or functional analogue thereof and an agent directed against disseminated intravascular coagulation. Such an agent may for example be a composition comprising heparin, however, in a preferred embodiment, the invention provides treatment with a hypotonic pharmaceutical composition comprising an NF-κB up-regulating peptide or functional analogue thereof. Such treatment may for example comprise infusions with Ringer's lactate for the first 24 hours, the Ringer's lactate provided with, preferably, 1-1000 mg/l NFκB regulating peptide such as VLPALPQ (SEQ ID NO: 9), GVLPALP (SEQ ID NO: 16) or MTRV (SEQ ID NO: 20), or mixtures of two or three of such peptides. At this stage, it is important to keep the volume up, and, if needed, provide the peptide or functional analogue thereof in even further hypotonic solutions, such as in 0.3 to 0.6% saline. NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended. It is preferred to monitor arachidonic acid metabolite and cytokine profiles, such as TNF-α, IL-10 levels, PGE2 and leukotriene levels in the plasma of the treated patient, and to stop treatment when these levels are considered within normal boundaries. In another embodiment, it is herein provided to modulate immunosuppression in a traumatized subject comprising providing the subject with a signaling molecule comprising a gene-regulatory peptide or functional analogue thereof wherein the subject is also provided with an agent directed against disseminated intravascular coagulation, in particular wherein the agent comprises Activated Protein C activity. Such an agent to modulate disseminated intravascular coagulation (DIC) comprises preferably (recombinant) human Activated Protein C. It is preferably given to the patient per infusionem, whereby NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended.

The invention provides a method for modulating an immunosuppression in a subject comprising providing the subject with a signaling molecule comprising a gene-regulatory peptide or functional analogue thereof, in particular wherein the signaling molecule up-regulates translocation and/or activity of a gene transcription factor, especially wherein the gene transcription factor comprises an NF-κB/Rel protein, particularly wherein translocation and/or activity of the NF-κB/Rel protein is increased. For anti-immunosuppressive treatment, it is preferred that the peptide is selected from the group of peptides having NFκB up-regulating activity in LPS unstimulated RAW264.7 cells, especially when the subject is at risk to experience a counter anti-inflammatory response syndrome, such as can seen to be occurring after severe trauma. Furthermore, a method is provided wherein the subject is also provided with an agent directed against disseminated intravascular coagulation, such as wherein the agent comprises Activated Protein C activity, or a similar anti-coagulant agent.

In response to a variety of pathophysiological and developmental signals, the NFκB/Rel family of transcription factors is activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFκB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or a modification or derivative thereof capable of modulating the activation of members of the NFκB/Rel family of transcription factors. Activation of NFκB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of genes that are under the control of NFκB/Rel family of transcription factors. Modulating comprises the upregulation or the downregulation of transcription. In a preferred embodiment, a peptide according to the invention, or a functional derivative or analogue thereof is used for the production of a pharmaceutical composition. Such peptides may be selected from group of peptides having NFκB down- or up-regulating activity in LPS stimulated RAW264.7 cells. More gene-regulating peptides and functional analogues can be found in a (bio)assay, such as a NFκB translocation assay as provided herein, and a by testing peptides for NFκB down- or up-regulating activity in LPS-stimulated or unstimulated RAW264.7 cells. Useful NFκB up-regulating peptides are VLPALPQ (SEQ ID NO: 9), GVLPALP (SEQ ID NO: 16) and MTRV (SEQ ID NO: 20). As indicated, more gene-regulatory peptides may be found with an appropriate (bio)assay. A gene-regulatory peptide as used herein is preferably short. Preferably, such a peptide is 3 to 15 amino acids long, more preferably, wherein the lead peptide is 3 to 9 amino acids long, most preferred wherein the lead peptide is 4 to 6 amino acids long, and capable of modulating the expression of a gene, such as a cytokine encoding gene, in a cell. In a preferred embodiment, a peptide is a signaling molecule that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-κB in an NF-κB assay, or AP-1 in an AP-1 assay, or by another method as provided herein. Fragments can be somewhat (i.e. 1 or 2 amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a signaling molecule capable of modulating gene expression. Of course, such replacement scanning or mapping can be undertaken with amino acids other than Ala as well, and also with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptides. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty. The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, Norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect. In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can for example be performed in the process of gene-regulatory peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV (SEQ ID NO: 1), AQGV (SEQ ID NO: 2), LQGA (SEQ ID NO: 3), VLPALP (SEQ ID NO: 4), ALPALP (SEQ ID NO: 5), VAPALP (SEQ ID NO: 6), ALPALPQ (SEQ ID NO: 7), VLPAAPQ (SEQ ID NO: 8), VLPALAQ (SEQ ID NO: 9), LAGV (SEQ ID NO: 10), VLAALP (SEQ ID NO: 11), VLPALA (SEQ ID NO: 12), VLPALPQ (SEQ ID NO: 13), VLAALPQ (SEQ ID NO: 14), VLPALPA (SEQ ID NO: 15), GVLPALP (SEQ ID NO: 16), VVCNYRDVRFESIRLPGCPRGVNPVVSYAVALSCQCAL (SEQ ID NO: 24), RPRCRPINATLAVEKEGCPVCITVNTTICAGYCPT (SEQ ID NO: 25), SKAPPPSLPSPSRLPGPS (SEQ ID NO: 26), LQGVLPALPQVVC (SEQ ID NO: 17), SIRLPGCPRGVNPVVS (SEQ ID NO: 27), LPGCPRGVNPVVS (SEQ ID NO: 18), LPGC (SEQ ID NO: 19), MTRV (SEQ ID NO: 20), MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the α-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/min; absorbance was detected from 190-370 nm. There were different gradient systems used. For example for peptides LQG and LQGV: 10 minutes 100% A followed by linear gradient 0-10% B in 50 minutes. For example for peptides VLPALP (SEQ ID NO: 6) and VLPALPQ (SEQ ID NO: 13): 5 minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO2 using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After 8 h of cultures cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 s, left standing at 4° C. for 15 min, and centrifuged at 15,000 rpm for 2 min. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 min on ice, then the lysates were centrifuged at 15,000 rpm for 2 min. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′) (SEQ ID NO: 28) synthesized to represent the NF-κB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 min at room temperature. The DNA-protein complex was resolved from free oligonucleotide by electrophoresis in a 6% polyacrylamide gel. The gels were dried and exposed to x-ray films.

The transcription factor NF-κB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-κB into the nucleus, on these extracts EMSA was performed. Here we see that indeed some peptides are able to modulate the translocation of NF-κB since the amount of labeled oligonucleotide for NF-κB is reduced. In this experiment peptides that show the modulation of translocation of NF-κB are: VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VLPALPQ (SEQ ID NO: 13), GVLPALP (SEQ ID NO: 16), VVC, MTRV (SEQ ID NO: 20), MTR.

RAW 264.7 mouse macrophages were cultured in DMEM, containing 10% or 2% FBS, penicillin, streptomycin and glutamine, at 37° C., 5% CO₂. Cells were seeded in a 12-wells plate (3×10⁶ cells/ml) in a total volume of 1 ml for 2 hours and then stimulated with LPS (E. coli 026:B6; Difco Laboratories, Detroit, Mich., USA) and/or NMPF (1 microgram/ml). After 30 minutes of incubation plates were centrifuged and cells were collected for nuclear extracts. Nuclear extracts and EMSA were prepared according to Schreiber et al. Cells were collected in a tube and centrifuged for 5 minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™Mini, Roche) and left on ice for 15 minutes. Twenty five micro liter 10% NP-40 was added and the sample was centrifuged (2 minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For Electrophoretic mobility shift assays an oligonucleotide representing NF-κB binding sequence (5′-AGC TCA GAG GGG GAC TTT CCG AGA G-3′) (SEQ ID NO: 28) was synthesized. Hundred pico mol sense and antisense oligo were annealed and labeled with γ-³²P-dATP using T4 polynucleotide kinase according to manufacture's instructions (Promega, Madison, Wis.). Nuclear extract (5-7.5 μg) was incubated for 30 minutes with 75000 cpm probe in binding reaction mixture (20 microliter) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, 2-4 hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptides are able to modulate the basal as well as LPS induced levels of NF-κB. In this experiment peptides that show the inhibition of LPS induced translocation of NF-κB are: VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VVC, MTR and circular LQGVLPALPQVVC (SEQ ID NO: 17). Peptides that in this experiment promote LPS induced translocation of NF-κB are: VLPALPQ (SEQ ID NO: 13), GVLPALP (SEQ ID NO: 16) and MTRV (SEQ ID NO: 20). Basal levels of NF-κB in the nucleus was decreased by VLPALPQVVC (SEQ ID NO: 21), LQGVLPALPQ (SEQ ID NO: 22), LQG and LQGV (SEQ ID NO: 10) while basal levels of NF-κB in the nucleus was increased by GVLPALPQ (SEQ ID NO: 23), VLPALPQ (SEQ ID NO: 13), GVLPALP (SEQ ID NO: 16), VVC, MTRV (SEQ ID NO: 20), MTR and LQGVLPALPQVVC (SEQ ID NO: 17). In other experiments, QVVC (SEQ ID NO: 29) also showed the modulation of translocation of NF-κB into nucleus (data not shown).

Further modes of identification of gene-regulatory peptides by NFκB analysis:

Cells: Cells will be cultured in appropriate culture medium at 37° C., 5% CO₂. Cells will be seeded in a 12-wells plate (usually 1×10⁶ cells/ml) in a total volume of 1 ml for 2 hours and then stimulated with regulatory peptide in the presence or absence of additional stimuli such as LPS. After 30 minutes of incubation plates will be centrifuged and cells are collected for cytosolic or nuclear extracts.

Nuclear Extracts: Nuclear extracts and EMSA could be prepared according to Schreiber et al. Method (Schreiber et al. 1989, Nucleic Acids Research 17). Cells are collected in a tube and centrifuged for 5 minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty five micro liter 10% NP-40 is added and the sample is centrifuged (2 minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′) (SEQ ID NO: 28) are synthesized. Hundred pico mol sense and antisense oligo are annealed and labeled with γ-³²P-dATP using T4 polynucleotide kinase according to manufacture's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 □l) containing 0.5 μg poly dI-dC (Amersham Pharmacia Biotech) and binding buffer BSB (25 mM MgCl₂, 5 mM CaCl₂, 5mM DTT and 20% Ficoll) at room temperature. Or cytosolic and nuclear extract from untreated cells or from cells treated with stimuli could also be incubated with probe in binding reaction mixture and binding buffer. The DNA-protein complex is resolved from free oligonucleotide by electrophoresis in a 4-6% polyacrylamide gel (150 V, 2-4 hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 miroliter Neutr-Avidin-plus beads for 1 h at 4° C. with constant shaking. Beads are washed five times with lysis buffer by centrifugation at 6000 rpm for 1 min. Proteins are eluted by incubating the beads in 0.05 N NaOH for 1 min at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-κB subunit are pooled and concentrated. Fractions are then dissolved in appropriate volume and could be analyzed on mass-spectrometry.

Further references: PCT International Patent Publications WO99/59671, WO01/72831, WO97/49721, WO01/10907, and WO01/11048, the contents of the entirety of all of which are incorporated by this reference.

What is claimed is:

1. A method for modulating or treating an immunosuppressive state in a subject comprising providing said subject with a gene-regulatory peptide or functional analogue thereof.

2. The method according to claim 1 wherein the subject has experienced trauma.

3. The method according to claim 1 wherein said peptide or analogue up-regulates translocation and/or activity of a gene transcription factor.

4. The method according to claim 2 wherein said peptide or analogue up-regulates translocation and/or activity of a gene transcription factor.

5. The method according to claim 3 wherein said gene transcription factor comprises an NF-κB/Rel protein.

6. The method according to claim 4 wherein said gene transcription factor comprises an NF-κB/Rel protein.

7. The method according to claim 1 wherein said gene-regulatory peptide or functional analogue thereof has NFκB up-regulating activity in LPS unstimulated RAW264.7 cells.

8. The method according to claim 2 wherein said gene-regulatory peptide or functional analogue thereof has NFκB up-regulating activity in LPS unstimulated RAW264.7 cells.

9. The method according to claim 3 wherein said gene-regulatory peptide or functional analogue thereof has NFκB up-regulating activity in LPS unstimulated RAW264.7 cells.

10. The method according to claim 4 wherein said gene-regulatory peptide or functional analogue thereof has NFκB up-regulating activity in LPS unstimulated RAW264.7 cells.

11. The method according to claim 5 wherein said gene-regulatory peptide or functional analogue thereof has NFκB up-regulating activity in LPS unstimulated RAW264.7 cells.

12. The method according to claim 6 wherein said gene-regulatory peptide or functional analogue thereof has NFκB up-regulating activity in LPS unstimulated RAW264.7 cells.

13. The method according to any one of claims 1 to 12 wherein the subject is at risk for suffering a counter anti-inflammatory response syndrome.

14. The method according to claim 13 wherein said gene-regulatory peptide or functional analogue thereof has NFκB up-regulating activity in LPS stimulated RAW264.7 cells.

15. The method according to claim 1 wherein the subject is also provided with an agent directed against disseminated intravascular coagulation.

16. The method according to claim 15 wherein said agent has Activated Protein C activity.

17. The method according to any one of claims 2 through 14 wherein the subject is also provided with an agent directed against disseminated intravascular coagulation.

18. The method according to claim 17 wherein said agent has Activated Protein C activity.

19. A pharmaceutical composition comprising:
an NF-κB up-regulating peptide or functional analogue thereof and
an agent directed against disseminated intravascular coagulation.

20. A hypotonic pharmaceutical composition comprising an NF-κB up-regulating peptide or functional analogue thereof.
